(19) United States
(12) Patent Application Publication
(10) Pub. No.: US 2025/0046399 A1
(43) Pub. Date: Feb. 6, 2025

(54) COMPRESSION TECHNIQUES FOR GENOMIC DATA

(71) Applicant: Illumina, Inc., San Diego, CA (US)

(72) Inventors: Guillaume Alexandre Pascal Rizk, Rennes (FR); Ali Chemkhi, Rennes (FR); Sergey Lobanov, Cambridge (GB)

(21) Appl. No.: 18/738,949

(22) Filed: Jun. 10, 2024

Related U.S. Application Data
(60) Provisional application No. 63/530,621, filed on Aug. 3, 2023.

(51) Int. Cl. G16B 50/50; G16B 30/10

(52) U.S. Cl. CPC G16B 50/50; G16B 30/10

(57) ABSTRACT

Methods, systems, and apparatus, including computer programs encoded on computer-storage media, for compressing genomic data. One of the methods includes obtaining a genomic data read; mapping the read to a plurality of different candidate reference genomes; selecting one of the candidate reference genomes based on the mapping; performing reference-based compression of the read using the selected reference genome; and storing the compressed genomic data read.

### CROSS-REFERENCE TO RELATED APPLICATIONS

This application claims priority of U.S. Provisional Application Ser. No. 63/530,621, filed Aug. 3, 2023. The contents of the prior application is incorporated herein by reference in its entirety.

### FIELD

This specification generally relates to compressing genomic data.

### BACKGROUND

Genomic read mapping is a computational process that aligns short deoxyribonucleic acid (DNA) sequences, known as reads, to a reference genome to determine their original genomic locations. DNA sequencing technologies generate millions of short reads, and read mapping is essential for understanding the structure and variation within the genome. During mapping, algorithms search for the best alignment between each read and the reference genome, taking into account factors like sequence similarity and alignment quality. By accurately mapping reads, researchers can identify genetic variants, analyze gene expression patterns, and investigate genomic features important for various applications, such as disease research, population genetics, and personalized medicine.

### SUMMARY

The present disclosure is generally directed towards the compression of genomic data, e.g., methylated or unmethylated DNA. When mapping a genomic read to a reference sequence, methylated or unmethylated nucleotides, such as adenine (A), thymine (T), guanine (G), and cytosine (C), can match with a single reference nucleotide. Because both methylated and unmethylated nucleotides can match to the same reference nucleotide, it can be difficult to determine whether a given read includes methylated or unmethylated nucleotides.

The present disclosure improves mapping technology by comparing an obtained data read to each of a plurality of different reference genomes. The plurality of reference genomes can include an unchanged reference genome, a reference genome that has been modified to convert C to T, and a reference genome that has been modified to convert G to A. Specific modifications of C to T and G to A can be useful when a read has been treated for methylation analysis. For example, a sample read can be treated using a bisulfite treatment. The bisulfite treatment converts unmethylated C to U which is read as T during PCR amplification, effectively converting C to T. The opposite strand, generated during PCR amplification, is read as A opposite the T, rather than a G in an unconverted sequence of the same strand, essentially converting G to A in the sequence read of the opposite strand. Detecting these conversions—e.g., using one or more modified reference genomes—can be used to identify a location or abundance of methylated C or the complementary G opposite a methylated C which is important for understanding gene regulation, epigenetics, and various biological processes.

DNA methylation is a biological process where methyl groups are attached to the DNA molecule, most commonly on the cytosine base, forming 5-methylcytosine. This process does not change the DNA sequence itself but will impact activity of the DNA segment where methylation occurs. This typically acts to repress gene transcription and can play a key role in many biological processes. Aberrant DNA methylation can lead to genomic instability and development of diseases such as cancer. Bisulfite sequencing is a method for detecting epigenetic methylation patterns at single-base resolution. This technique involves chemically treating DNA with sodium bisulfite, which converts unmethylated cytosine bases to uracil, but does not alter methylated cytosines. When sequenced, non-methylated cytosine will be read as thymine (T). Subsequent bioinformatics pipelines will be able to infer methylation at base resolution by comparing sequencing results and original DNA sequence.

The impact of methylation in diseases development can lead to large volumes of whole genome bisulfite sequencing (WGBS) data being generated. Furthermore, genomic data often needs to be stored for many years for research purposes or clinical regulatory requirements, leading to very high storage costs. The present disclosure aims at compressing genomic data—e.g., in FASTQ format-generated by bisulfite sequencing, to decrease the storage requirements and cost.

Techniques described in this document include selecting a reference genome from the plurality of different reference gnomes for use in compressing the obtained read using reference-based compression.

In some implementations, techniques described increase a compression ratio for storing genomic data read mappings. For example, techniques can include generating a set of multiple reference sequences, identifying the reference sequence that most closely matches a genomic data read to be mapped, and using the identified reference sequence to compress and store the genomic data read. Because storage compression can be proportional to the number of mismatches between a read and a reference, by reducing the number of mismatches between the read and reference, the techniques can increase a compression ratio for storing read mappings.

In some implementations, techniques described are applied to methylation data. For example, methylated DNA and unmethylated DNA can have a number of mismatches in their nucleotide sequences. By generating multiple references, include references for methylated DNA and references for unmethylated DNA, the number of mismatches stored can be reduced and resulting required memory for storage can be reduced.

Compressed genomic data read mappings can be used in various analysis steps, including analysis for disease detection and diagnoses. DNA methylation can refer to the addition of methyl groups to cytosine bases in DNA. Methylated DNA and unmethylated DNA differ based on the presence or absence of these methyl groups. The patterns of DNA methylation can play a role in gene regulation and are associated with various diseases, including cancers such as liver cancer. Aberrant DNA methylation patterns can be used as biomarkers for medical diagnostics, aiding in detection and diagnosis. Methylation changes in specific genes can indicate the presence of cancer while the analysis of methylation patterns can provide prognostic information and guide treatment decisions. DNA methylation analysis can be performed non-invasively using bodily fluids, allowing for early detection and monitoring of liver cancer without the need for invasive procedures.

In general, one aspect of the subject matter described in this specification can be embodied in methods that include the actions of obtaining a genomic data read; mapping the read to a plurality of different candidate reference genomes; selecting one of the candidate reference genomes based on the mapping; performing reference-based compression of the read using the selected reference genome; and storing the compressed genomic data read.

Other implementations of this aspect include corresponding computer systems, apparatus, computer program products, and computer programs recorded on one or more computer storage devices, each configured to perform the actions of the methods. A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

The foregoing and other implementations can each optionally include one or more of the following features, alone or in combination. In some implementations, the plurality of different candidate reference genomes includes two or more candidate reference genomes that are modifications of a single genomic reference. In some implementations, the plurality of different candidate reference genomes comprises a candidate reference genome that includes nucleotide sequences of a methylome. In some implementations, the candidate reference genome that includes nucleotide sequences of the methylome includes portions of the methylome where cytosine is converted to thymine. In some implementations, the portions of the methylome where cytosine is converted to thymine are not CpG regions. In some implementations, the candidate reference genome that includes nucleotide sequences of the methylome includes portions of the methylome where guanine is converted to adenine. In some implementations, the portions of the methylome where guanine is converted to adenine are not CpG regions. In some implementations, storing the compressed genomic data read comprises: storing an indication of a number of mismatches between nucleotides of the read and the selected reference genome.

In some implementations, storing the compressed genomic data read comprises: storing an indication of an offset that indicates a position of the genomic data read relative to the selected reference genome. In some implementations, the offset indicates a number of nucleotides from a beginning of the selected reference genome to a start of the genomic data read. In some implementations, actions include generating a combined reference using the plurality of different candidate reference genomes, wherein the offset is equal to an offset between the genomic data read and the selected reference genome plus a length of one or more of the plurality of different candidate reference genomes.

In some implementations, selecting the one of the candidate reference genomes based on the mapping comprises: determining a number of mismatches between the genomic data read and the plurality of different candidate reference genomes. In some implementations, actions include selecting the selected reference genome based on a minimum mismatch value from the number of mismatches between the genomic data read and the plurality of different candidate reference genomes.

In some implementations, storing the compressed genomic data read comprises: generating encoded data representing the genomic data read; and storing the encoded data representing the genomic data read in a hash table.

In some implementations, actions include storing data indicating an associated methylation state for one or more variants, the one or more variants determined based on mapping the read to at least one additional reference genome. In some implementations, storing the data indicating the associated methylation state for the one or more variants includes storing the data indicating the associated methylation state with data indicating the one or more variants in a single data structure.

Advantageous implementations can include one or more of the following features. For example, by using a selected reference based on a set of one or more modified reference genomes, techniques described in this document can improve compression ratios of gene read mapping by a factor of 2 or more (e.g., 1.8 compression ratio using standard reference genome compared to compression ratios of 3.5 to 4.0 using modified reference techniques described in this document).

The details of one or more embodiments of the invention are set forth in the accompanying drawings and the description below. Other features and advantages of the invention will become apparent from the description, the drawings, and the claims.

### BRIEF DESCRIPTION OF THE DRAWINGS

FIG. 1 is a diagram showing an example of a system for compressing genomic data.

FIG. 2 is a flow diagram illustrating an example of a process for compressing genomic data.

FIG. 3 is a flow diagram illustrating an example compression process.

FIG. 4 is a diagram showing an example of comparing genomic data reads to references.

FIG. 5 is a diagram showing an example variant call format (VCF) code snippet.

Like reference numbers and designations in the various drawings indicate like elements.

### DETAILED DESCRIPTION

In general, compression ratios for mismatch based alignment can be proportional to the number of mismatches in the alignment, with a perfect alignment (no mismatches) yielding the best compression ratio.

One challenge with WGBS data is that because of bisulfite treatment, the non-methylated C will be read as T by the sequencer and will generate many mismatches between read sequence and reference genome (e.g., in every position where the reference is C and the bisulfite treated sequence includes non-methylated C, now read as T), leading to alignments with low similarity and high mismatches. The high number of mismatches reduces the compression ratio and increases storage requirements and costs for storing resulting alignment data.

To improve compression—e.g., on bisulfite sequencing data—techniques described in this document describe using a set of modified references (e.g., one copy of the human reference with C converted to T; one copy of the human reference with G converted to A; one copy of the methylome unchanged (where methylome is a subset of a reference genome where methylation is expected to occur); one copy of the methylome with C converted to T unless it lies in CpG region; one copy of the methylome with G converted to A unless it lies in CpG region).

Compression improvements of the techniques described in this document are shown in the following table (data set: Homo sapiens Bisulfite-sequence, 43.2 gigabases (1 gigabase=1 billion bases)):

| | Compression | |
|---|---|---|
| | ratio | Compression time (s) |
| Standard | 1.7 x | 295 s |
| unmodified | | |
| Reference | | |
| Modified Bisulfite | 3.8 x | 244 s |
| Reference | | |
| Spring | 2.4 x | 3785 s |

In reference to the above table, compression ratio is defined as: size of original file in fastq.gz divided by the size of compressed file. Compression time is the real time returned by the ‘time’ command. All programs are run with 16 threads. Spring represents a generic compression algorithm used for comparison purposes (Spring version 1.1.1). The table shows that the techniques described, corresponding to “Modified Bisulfite Reference” results in a compression ratio roughly 2×that of the “Standard unmodified Reference” which can be computed in less time (244 seconds compared to 295 seconds).

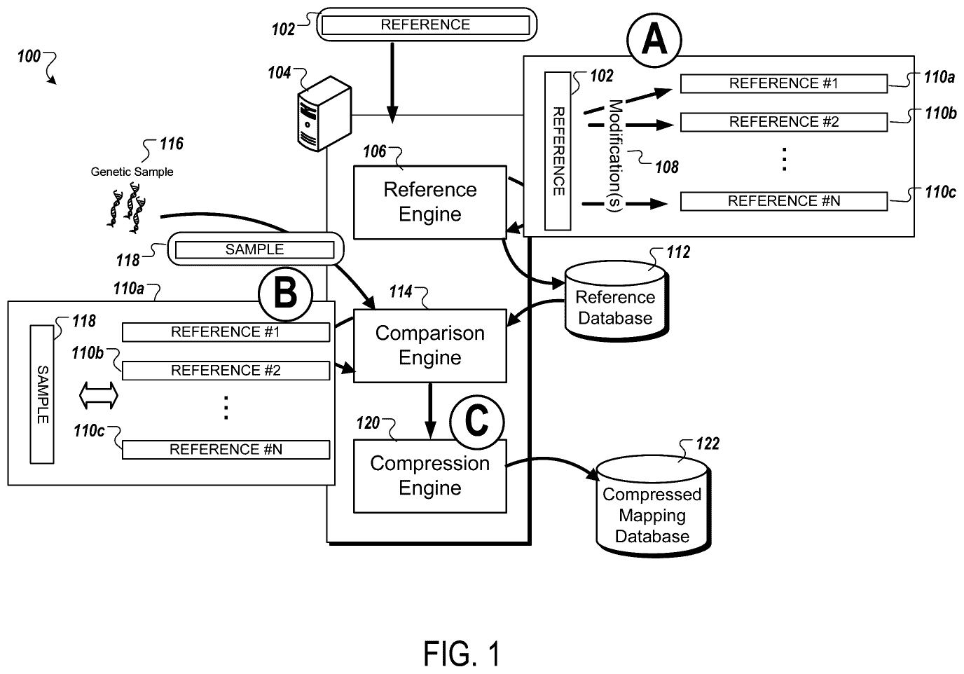

FIG. 1 is a diagram showing an example of a system 100 for compressing genomic data. The system 100 includes a control unit 104. The system 100 generates modified references 110a-c from a genomic reference 102 (e.g., a known human genome) and the uses the modified references 110a-c to generate a compressed mapping for the genetic sample 116. Although described in reference to human genomic sequences, the techniques described can be applied to various non-human genomic samples or references, e.g., Sus scrofa (pig), Gallus gallus (chicken), Oryza sativa (Japanese rice), Arabidopsis thaliana, Triticum aestivum (bread wheat), Bos taurus (cattle), Glycine max (soybean), Rattus norvegicus (norway rat), Zea mays (maize), Danio rerio (zebrafish), Mus musculus (house mouse), Caenorhabditis elegans (roundworm).

In stage A, the control unit 104 obtains the reference 102. The reference 102 can include a sequence of nucleotides corresponding to a known human genome. A reference engine 106 of the control unit 104 can generate the modified references 110a-c. Although three modified references are shown in the example of FIG. 1, other numbers of modified references could be generated.

In some implementations, the modified references 110a-c include 5 modified references. In general, the reference engine 106 can perform any number of modifications 108 to generate any number of references modified from the reference 102. For example, the modified references 110a-c can include a modified reference where the reference engine 106 converts C nucleotides of the reference 102 to T nucleotides. The modified references 110a-c can include a modified reference where the reference engine 106 converts G nucleotides of the reference 102 to A nucleotides. Using modified references that include conversions from C to T and G to A can be useful in determining whether or not a sample read (e.g., treated using a bisulfite treatment) includes methylated C. For example, a treated sample with methylated C at a given location along a read can match with a reference modified by converting C to T but not match an unmodified reference at the given location. By analyzing one or more mismatches, the control unit 104—or analyzing engine communicably connected to the control unit 104 and configured to compressed mapping output from the control unit 104—can identify locations or abundance of methylated nucleotides (e.g., C and G).

The modified references 110a-c can include a modified reference where the reference engine 106 extracts only known methylome regions—e.g., regions of DNA where methylation (the addition of a methyl group to the DNA molecule, typically at cytosine residues in a CpG dinucleotide context) is expected to occur—from the reference 102. In some implementations, the reference engine 106 does not convert any nucleotides in the extracted methylome regions. The modified references 110a-c can include a modified reference where the reference engine 106 converts C nucleotides in extracted methylome regions to T nucleotides. The modified references 110a-c can include a modified reference where the reference engine 106 converts G nucleotides in extracted methylome regions to A nucleotides. In some implementations, the reference engine 106 only converts C nucleotides to T nucleotides or G nucleotides to A nucleotides if the original nucleotide is not in a CpG region—e.g., C or G nucleotide adjacent to a G or C nucleotide, respectively. A CpG region can refer to a C nucleotide directly adjacent to a G nucleotide, or vice versa. A CpG region can refer to a C nucleotide separated by one or more nucleotides from a subsequent G nucleotide, or vice versa. A CpG region can refer to a C nucleotide and a G nucleotide on a same strand. A CpG region can include a genomic sequence that includes one or more instances of C followed by G. These regions are often associated with a potential for DNA methylation.

In some implementations, the reference engine 106 stores the modified references 110a-c in the reference database 112. The reference database 112 can include one or more computer readable memory storage devices on one or more computers performing operations of the control unit 104 or communicably connected to the control unit 104.

In stage B, the comparison engine 114 compares the sample 118 to the references 110a-c. For example, the comparison engine 114 can identify one or more nucleotides of the sample 118 and compare the one or more nucleotides of the sample 118 with one or more nucleotides of the references 110a-c. The sample 118 can be a sequenced set of nucleotides from a sequencer processing the genetic sample 116. The sample 118 can be a modified version of an original nucleotide sequence of the genetic sample 116—e.g., a modified version resulting after a bisulfite treatment to convert unmethylated cytosine or a polymerase chain reaction (PCR) amplification.

In some implementations, the comparison engine 114 determines a comparison that results in a fewest number of mismatches. For example, the comparison engine 114 can record a number of mismatches for comparing nucleotides of the sample 118 to nucleotides of the references 110a-c. In cases where the references 110a-c include a number of references (e.g., 5 references), the comparison engine 114 can generate that number of mismatch values indicating a number of mismatches between nucleotides of the sample 118 and the references 110a-c at one or more locations along positions of DNA.

The comparison engine 114 can identify the reference of the references 110a-c that corresponds to a lowest number of mismatches and the compression engine 120 can use mismatches from that reference to store in the compressed mapping database 122—e.g., in a data structure that captures variations between the sample 118 and a selected reference of the references 110a-c. In some implementations, a data structure used to store a compressed mapping includes a value indicating a reference offset. The reference offset can indicate which of the one or more references used—e.g., the references 110a-c—were mapped to the given compressed data read. Because a compressed version of the mapping will store data indicating a number of mismatches, by selecting the reference with the least number of mismatches, the system 100 can reduce the data required for storing and therefore reduce the compressed size of the data and required memory storage. This reduction can also speed up the act of transferring the data to storage or retrieval and other post storing processes, such as analysis—e.g., for medical diagnosis or testing.

In some implementations, the comparison engine 114 generates a single reference from the references 110a-c. For example, the comparison engine 114 can combine the references 110a-c to generate a single reference where the single reference includes each of the references 110a-c appended to one another. In some implementations, the comparison engine 114 generates one or more references from the references 110a-c, or a combination of one or more references from the references 110a-c, as a hash table. For example, the comparison engine 114 can separate the references 110a-c into one or more k-mers (e.g., nucleotide sequence of length=k). The length of k-mer can be adjusted to help prevent false positives or noise in mapping—e.g., longer sequences to account for information being destroyed in chemical processes to convert one or more nucleotides to one or more other nucleotides, such as C to T or G to A. In some implementations, k is equal to 27.

In some implementations, the comparison engine 114 generates a hash table representing one or more references. For example, the comparison engine 114 can generate a hash table representing a combination of the references 110a-c. The hash table representing the combination of the references 110a-c can include data indicating nucleotide sequences (e.g., k-mers) of the references 110a-c. In some implementations, the hash table representing the combination of the references 110a-c includes multiple hash values generated using one or more hash functions. For example, the comparison engine 114 can generate one or more hash values using one or more hash functions for each k-mer extracted from one or more of the references 110a-c or a combined version of the references 110a-c used as a single reference.

In some implementations, the comparison engine 114 generates a hash table item for one or more k-mers extracted from one or more of the references 110a-c or a combined version of the references 110a-c used as a single reference. For example, the comparison engine 114 can combine the references 110a-c to generate a combined reference. In some implementations, the comparison engine 114 uses the combined reference to generate a hash table representing the combined reference. Using a hash table for comparison can help improve efficiency of comparing the sample 118 to the references 110a-c (e.g., compared to comparing binary sequences, which can be billions of bases long, representing the nucleotide sequence of each). Using a hash table representation can improve access and read times necessary to compare a sample (e.g., the sample 118) with a reference (e.g., the references 110a-c).

In some implementations, the comparison engine 114 generates a hash table using one or more hash functions. For example, the comparison engine 114 can generate a hash table value using a first hash table function applied to one or more k-mers extracted (e.g., by the comparison engine 114). The comparison engine 114 can use the generated hash table values to store data corresponding to the corresponding extracted k-mer(s) used to generate the hash value in the generated hash table. In some implementations, a hash table value represents an index in the hash table. The comparison engine 114 can store data corresponding to the given k-mer, such as (i) a signature of a k-mer generated by a second hash function (e.g., different than the first hash table function where the signature represents a hash value of the nucleotide sequence used to generate the corresponding hash table value) or (ii) one or more locations of the k-mer occurrence within a genomic read, such as a selected reference of the references 110a-c or a location along a combination of the references 110a-c.

In some implementations, the comparison engine 114 generates one or more keys for a hash reference table using a hash signature function. For example, the comparison engine 114 can generate a signature value for one or more k-mers of the references 110a-c. In some implementations, the signature generated by the comparison engine 114 is a compressed version of one or more k-mers. For example, a k-mer can be represented, as a sequence of nucleotides, in 16 bits for a k=16 k-mer with 16 nucleotides. The signature generated by the comparison engine 114 and a hash signature function can be represented by 8 bits. This key compression can significantly reduce a required size of the reference hash table. The decreased size can decrease look up time, storage time, and improve overall performance.

In some implementations, the comparison engine 114 extracts one or more k-mers from the sample 118 and compares the one or more k-mers to one or more of the references 110a-c. For example, the comparison engine 114 can use a hash function to generate a hash of one or more k-mers extracted from the sample 118. The hash can match one or more hashes stored in a hash table generated by the comparison engine 114. The hash value can correspond to a particular reference (e.g., one of the references 110a-c). The comparison engine 114 can compare a nucleotide sequence of the sample 118 and a nucleotide sequence of the particular reference by aligning the two sequences such that the matched k-mer used to compute the matching hash aligns in the two sequences. The reference of the references 110a-c corresponding to the alignment that results in the fewest mismatches can be selected as the reference with which to store the mapping of the sample 118.

In some implementations, selecting one of the references 110a-c based on the mapping includes determining a location along a combined reference as the alignment for a sample read—e.g., the sample 118. For example, one can imagine lining up all the references 110a-c end to end to create a larger reference. Although a system can be configured to compare a sample read with such a generated binary, linear, representation of a reference, it is more computationally and energy efficient to generate a hash table as described in this document. The hash table can be generated using a combined version of the references 110a-c—e.g., a modified reference that lines up all the references 110a-c end to end to create a much longer reference string. By using a combined reference, the system 100 can determine an offset for mapping the sample 118 to the combined reference where the offset also indicates what reference of the multiple references 110a-c (included in the combined reference) a read of the sample 118 most closely matches—e.g., indicating one or more locations of methylated C in the sample 118 when the read most closely matches a reference not including transformations from C to T and the read was treated using a bisulfite treatment because methylated C does not convert to T after a bisulfite treatment but unmethylated C does.

In stage C, the compression engine 120 generates a compressed mapping of the sample 118 using the references 110a-c. In some implementations, the compression engine 120 obtains information indicating a reference selected by the comparison engine 114 and a number of mismatches between the selected reference and the sample 118. The compression engine 120 can store the mapping of the sample 118 to the selected reference in a compressed format—e.g., a hash table. The compressed format can include one or more values indicating an offset value. The offset value can indicate a position of the sample 118 relative to the selected reference—e.g., a number of nucleotides from a beginning of a reference to the start of the sample 118.

By creating a single reference, the compression engine 120 can store a single offset value instead of additional values for offset and an index indicating which reference to use. By storing a single value, the compression engine 120 can help reduce the amount of data stored in the compressed mapping database 122 indicating a mapping of the sample 118.

The single offset value can indicate a number of nucleotides from a beginning of a selected reference to the start of the sample 118 plus any modified reference appended at the beginning of the selected reference to create the single reference. For example, if the selected reference is the first reference combined in a single reference, the offset would be the same in an implementation that used a single combined reference as an implementation that used multiple references and a data value that indicated which reference is used. However, if the selected reference was the second combined reference in a single reference, the offset would include the offset for the selected reference plus the number of nucleotides in the first reference situated prior to the selected reference in the single generated reference.

In some implementations, the compression engine 120 generates a hash table to compress a mapping of the sample 118. The hash table can include a number of different hash table entries for mapping portions of the sample 118 to portions of the reference selected by the comparison engine 114. The hash table can include a key value generated by hashing a portion of the sample 118 or the selected reference.

In some implementations, a mapping of the sample 118 includes an indication of one or more references used to compress the sample 118. For example, the compression engine 120 can include an indication of the set of references 110a-c used to generate a compressed version of the mapping of the sample 118 to facilitate accurate decompression (e.g., accurate decompression uses the same reference data as used for compression). In some implementations, an indication of one or more references used to compress the sample 118 includes a unique set of one or more characters. For example, for decompression, a decompression device—e.g., the control unit 104 or other device—can obtain a set of unique characters stored with the compressed mapping of the sample 118. The unique characters can indicate the references 110a-c used for compression. The decompression device can use the unique characters to obtain reference data—e.g., from a connected server—to enable the decompression device to decompress the compressed mapping of the sample 118. In some implementations, a device for providing references and checking unique characters uses one or more checksum algorithms—e.g., Message Digest Algorithm (MD) 5—to determine whether or not a given reference set matches a reference set used to compress data.

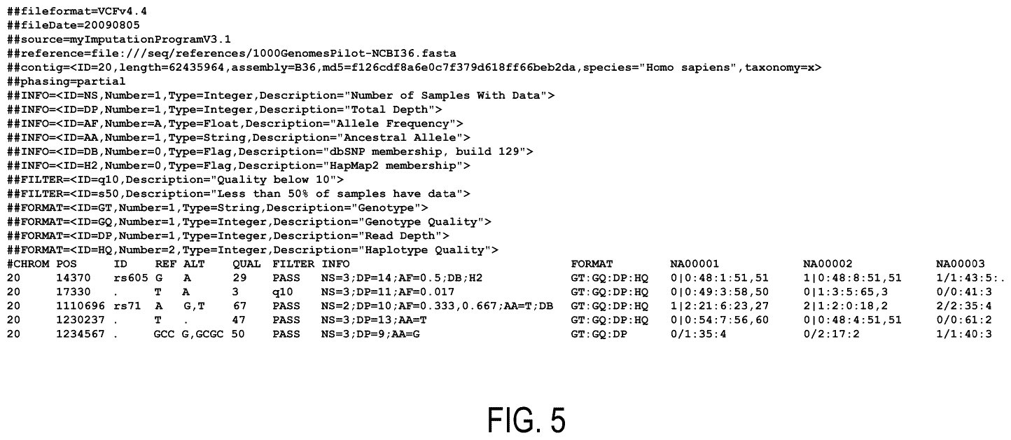

In some implementations, mappings data is stored in a variant call format (VCF). An example VCF code snippet is shown in FIG. 5. The comparison engine 114 can determine one or more mismatches between nucleotides of the sample 118 and the references 110a-c. These mismatches can be included as one or more variants in a VCF. In some cases, an enhanced VCF can be used to store variant data. For example, a VCF can be used that stores each variant detected as a row in a data structure. The VCF can include one or more tags to identify whether or not a variant corresponds to a specific type of methylation, e.g., 5-methylcytosine, 5-hydroxymethylcytosine, N6-methyladenine, N4-methylcytosine, or a combination of these among others. Prior VCF structures do not include methylation data and require an additional reporting structure that reports methylation level only for homozygous reference cytosines. Putting this additional methylation information into the VCF file allows reporting of methylation level for cytosines (e.g., homozygous reference cytosines and cytosines at Single Nucleotide Variants, Insertions, Deletions, or a combination of these among others) with additional possibility to report allele-specific methylation. In some cases, previous additional reporting structures duplicate genetic locating information associated with a mapped sequence.

FIG. 1 is described with reference to stages A through C. Although described in order from stage A through C, operations of the system 100 can occur in different orders or in parallel. For example, the control unit 104 can obtain one or more other reference sequences and process comparisons or compressions of other samples either in parallel with or prior to mapping the sample 118.

As used herein, the term “engine” refers to a set of computing/programmatic instructions, that when executed by a data processing apparatus (e.g., a processor, such as the control unit 104 or a machine communicably connected to the control unit 104), result in the performance of certain operations associated with the underlying component (e.g., the reference engine 106, the comparison engine 114, and the compression engine 120).

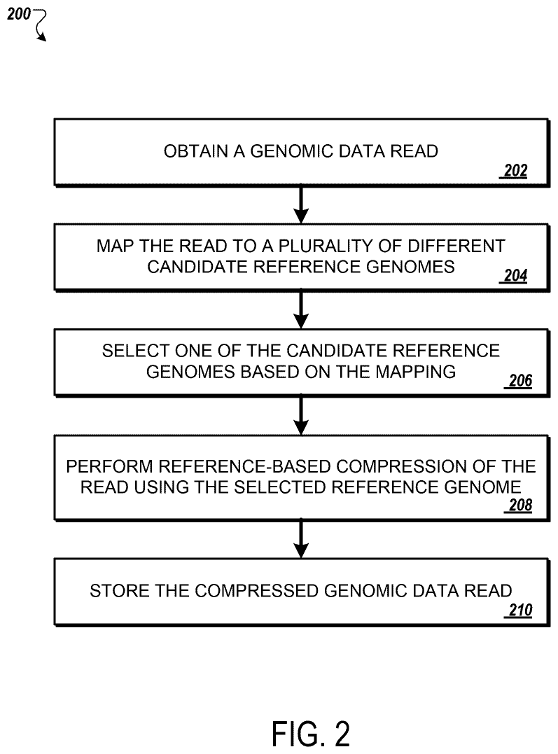

FIG. 2 is a flow diagram illustrating an example of a process for compressing genomic data. The process 200 may be performed by one or more electronic systems, for example, the system 100 of FIG. 1.

The process 200 includes obtaining a genomic data read (202). For example, control unit 104 obtains the sample 118. The sample 118 can be a genomic data read sequenced from a genetic sample 116 (e.g., human tissue, hair, among others). The sample 118 can be sequenced by another machine communicably connected to the control unit 104. The other machine can transmit data indicating a nucleotide sequence of the sample 118 via wired or wireless communication networks to the control unit 104.

The process 200 includes mapping the read to a plurality of different candidate reference genomes (204). For example, the comparison engine 114 can compare the sample 118 to the references 110a-c. In some implementations, the comparison engine 114 uses a hash table of one or more of the references 110a-c to compare the sample 118 with the references 110a-c. In some implementations, the comparison engine 114 directly compares one or more nucleotides of the one or more of the references 110a-c with one or more nucleotides of the sample 118.

The process 200 includes selecting one of the candidate reference genomes based on the mapping (206). For example, the comparison engine 114 can select one of the references 110a-c. The comparison engine 114 can select one of the references 110a-c based on a number of mismatches between nucleotides of the references 110a-c and nucleotides of the sample 118. In some implementations, the comparison that yields the fewest mismatches is selected by the comparison engine 114 as the reference for mapping storage.

The process 200 includes performing reference-based compression of the read using the selected reference genome (208). For example, the compression engine 120 generates a compressed version of a mapping between the sample 118 and one or more of the references 110a-c. In some implementations, the compressed version is stored in a data structure that captures variations between the sample 118 and a selected reference of the references 110a-c.

The process 200 includes storing the compressed genomic data read (210). For example, the compression engine 120 can store a compressed mapping of the sample 118 mapped to one of the references 110a-c in the compressed mapping database 122. The data size of the compressed form used can be less than storing a reference-based mapping using another reference-such as a standard known genome. For example, by using modified references 110a-c, the system 100 can reduce storage required to store compressed reference-based mappings of genomic data.

Depending on an exact library protocol, the system 100 can use different read alignments with certain combinations of three factors: (i) Reference base conversions (C→T or G→A) (ii) Read base conversions (C→T or G→A) (iii) Alignment orientation allowed (forward or reverse-complemented (RC)). Since the two mates in paired-end libraries are read in opposite directions, the latter two factors (read conversion and alignment orientation) have the opposite settings for the two mates. Below is a table showing the alignment types required by three different methyl-seq protocols which can be used with the compressed mappings generated by the system 100. Mappings of references and reads, indicated below, can be mapped as described in this document:

| | | | | | Orientation |
|---|---|---|---|---|---|
| Protocol | BAM | Reference | Read 1 | Read 2 | Constraint (R1) |
| Directional | | | | | |
| | 1 | C->T | C->T | G->A | Forward-only |
| | 2 | G->A | C->T | G->A | RC-only |
| Non-Directional, | | | | | |
| Or Directional- | | | | | |
| Complement | | | | | |
| | 1 | C->T | C->T | G->A | Forward-only |
| | 2 | G->A | C->T | G->A | RC-only |
| | 3 | C->T | G->A | C->T | RC-only |
| | 4 | G->A | G->A | C->T | Forward-only |
| PBAT | | | | | |
| | 3 | C->T | G->A | C->T | RC-only |
| | 4 | G->A | G->A | C->T | Forward-only |

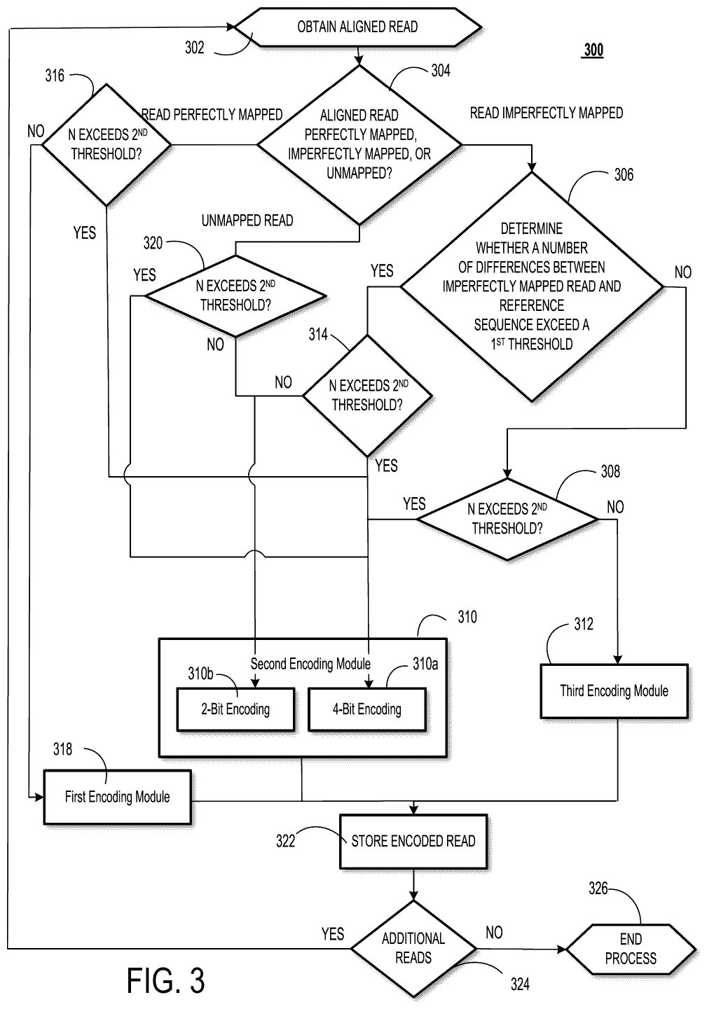

An example of compression performed by the compression engine 120 is shown in FIG. 3. Execution of the compression method 300—e.g., by the compression engine 120—can begin with an initial stage 302 that includes obtaining an aligned read record (also referred to below as “obtained read record” or “unmapped read”/“mapped read”/“perfectly mapped read”/“imperfectly mapped read” based on the obtained read record's subsequent classification during execution of method 300). In some implementations, the aligned read record can be obtained from a plurality of aligned read records that are stored in a manner so that their initial order as provided by the sequencing device is preserved. Thus, the entire operation of the mapping and aligning module and the compression module can keep the read records in their initial order as provided by the sequencing device. In some implementations, the aligned read records can be stored to preserve their initial order by using a sequence_id that is stored with each aligned read record and incremented with each aligned read record produced by the mapping and aligning module. In some implementations, an aligned read record is obtained from the comparison engine 114—e.g., a read of the sample 118 aligned to one or more of the references 110a-c.

The method 300 includes determining whether a read corresponding to the aligned read record is perfectly mapped, imperfectly mapped, or unmapped at stage 304.

The compression method of the present disclosure can include a next stage 304 of determining—e.g., by the compression engine 120—whether the obtained read record corresponds to a read that is perfectly mapped with the reference sequence, imperfectly mapped with the reference sequence, or unmapped with the reference sequence. In some implementations, the compression engine 120 determines whether the read is perfectly mapped, imperfectly mapped, or unmapped read based on information received from the comparison engine 114, which can include a mapping and aligning module. This information can include information such as, for example, whether the read represented by the obtained read record was mapped or unmapped, whether the read represented by the read record is perfectly mapped or imperfectly mapped, an indication of a number of total mismatches such as variants or sequencing errors, undetermined bases, or any combination thereof. In some implementations, this information can be included within the read record itself.

In some implementations, the compression engine 120 may first determine at stage 304 whether the aligned read was mapped or unmapped. If the compression engine 120 determines that the aligned read was unmapped, then the compression engine 120 can continue execution of the process 300 of FIG. 3 at stage 320. If the compression engine 120 determines that the read was mapped, then the compression engine 120 can further determine at stage 304 if the read was imperfectly mapped or perfectly mapped.

In some implementations, the compression engine 120 determines at stage 304 if a read was imperfectly mapped or perfectly mapped by evaluating a number of mismatches in the read. In some implementations, the number of mismatches can be provided by mapping and aligning module of the comparison engine 114 and obtained from a read record provided by the comparison engine 114. The number of mismatches may be tallied in different ways for different implementations. In some implementations, the number of mismatches at stage 304 may not include a number of undetermined bases N. In other implementations, the number of mismatches determined at stage 304 may include a total of the number of mismatches and a number of undetermined bases N.

In the example of FIG. 3, it is assumed that an undetermined base N is not a mismatch. As a result, a perfectly mapped read may include 0 mismatches and one or more undetermined bases N. Thus, an imperfectly mapped read, in this implementation, would need to have at least one mismatch, and may or may not have any undetermined bases N. However, in other implementations, the process 300 of FIG. 3 can be modified by assuming that the presence of an N in a read could be a mismatch. In such implementations, a read could be determined to be a perfectly mapped read only if the read is determined to have 0 mismatches and 0 undetermined bases N, with a read having 0 mismatches and one or more undetermined bases N being classified as an imperfectly mapped read.

In a first implementation, at stage 304, if the compression engine 120 determines that the total number of mismatches is equal to zero and the total number of undetermined bases N is zero or more, then the compression engine 120 can determine at stage 304 that the obtained aligned read is a perfectly mapped read and can continue execution of the process 300 of FIG. 3 at stage 316. Alternatively, in this first implementation if at stage 304, the compression engine 120 determines that the total number of mismatches is greater than zero and the total number of undetermined bases N is zero or more, then the compression engine 120 can determine, during stage 304, that the read corresponding to the obtained read record is an imperfectly mapped read and the compression engine 120 can continue execution of the process 300 of FIG. 3 at stage 306.

In a second and alternative implementation, at stage 304, the compression engine 120 will only determine that the read is a perfectly mapped read if the total number of mismatches is equal to zero and the total number of undetermined bases N is zero, and in such a scenario, the compression engine 120 can continue execution of the process 300 of FIG. 3 at stage 316. Alternatively, in this second implementation if at stage 304, the compression engine 120 determines that the total number of mismatches is greater than zero or the total number of undetermined bases N is greater than zero, then the compression engine 120 can determine, at stage 304, that the read corresponding to the obtained read record is an imperfectly mapped read and the compression engine 120 can continue execution of the process 300 of FIG. 3 at stage 306.

However, it is noted that the above implementations are merely examples as to how the compression engine 120 can determine at stage 304 that a read corresponding to the obtained read record is perfectly mapped, imperfectly mapped, or unmapped. For example, in some implementations, such a determination can instead be made based on information contained in the obtained read record and without comparison of a number of mismatches to a threshold, without a comparison of a number of undetermined bases N to a threshold, or both. By way of example, the read record can maintain bit flags in the header, or other portion, of the read record that indicates whether the read is mapped or unmapped, perfectly mapped, imperfectly mapped, or the like. In such implementations, the compression engine 120 can make a determination at stage 304 as to whether the aligned read record is mapped, unmapped, perfectly mapped, or imperfectly mapped based on the bit flags of the read record without comparison of a number of mismatches or undisclosed bases N to thresholds. Other implementations also fall within the scope of the present disclosure. For example, it is conceivable that implementations can be employed where information that is stored in a data structure that is different than the read record can be accessed and considered to read bit flags, or other data, to indicate whether a read corresponding to the particular read record is mapped, unmapped, perfectly mapped, or imperfectly mapped.

### “Read Imperfectly Mapped” Branch of Stage 304

If the compression engine 120 determines at stage 304 that the read corresponding to the obtained read record—e.g., a read record generated by the comparison engine 114 using the sample 118 and the references 110a-c and obtained by the compression engine 120—is an imperfectly mapped read, then the compression engine 120 can determine, at stage 306, whether a number of differences between said imperfectly mapped read and the reference sequence exceeds a first threshold value. This can include a total number of mismatches, with the total number of mismatches including a summation of any difference between the aligned read and the reference sequence including variants, sequencing errors, and undetermined bases N. In other implementations, the number differences at stage 306 may include only a number of mismatches without factoring in the number of undetermined bases N. In some implementations, the number of mismatches can be provided by a mapping and aligning module of the comparison engine 114 and obtained from the read record.

In some implementations, the first threshold value can be 31. This specific value can be chosen so as to provide the best possible compromise for storing the number of mismatches in a sufficiently compact manner, as will be better understood later with regard to subsequent stages. Indeed, it has been statistically observed that in a vast majority of the cases, the imperfectly mapped reads have less than 31 mismatches. The principle lying behind that choice consists in encoding in the most compact way the most frequent cases, leaving some very few degraded cases. However, though there are particular advantages that can be achieved using a first threshold value of 31, the present disclosure is not limited to only those implementations where the first threshold value is equal to 31. Instead, for other implementations it may be desirable to use a threshold value higher than 31. For example, while aspects (e.g., threshold value of 31 mismatches) may be intended for use compressing read records representing reads generated by short read sequencers, it is contemplated that the genomic data compression methods of the present disclosure can be used in other implementations such as to compress read records generated by long read sequencers. Thus, in such implementations, where reads are represented by read records that are significantly longer than 350 nucleotides or bases in length, the threshold value can be set to a higher value than 31 to enable functionality of the compression methods of the present disclosure for long read systems.

### “YES” Branch of Stage 306

If the compression engine 120 determines at stage 306 that the number of differences between the imperfectly mapped read and the reference sequence exceeds the first threshold, then the compression engine 120 can continue execution of the process 300 at stage 314. At stage 314, the compression engine 120 can determine whether a number of undetermined bases “N” in the imperfectly mapped read exceeds a second threshold value. In some implementations, the second threshold value can also be equal to 31. However, like the first thresholds, the second threshold value of the present disclosure is not limited to a value of 31. Instead, any number value, including values higher or lower than 31, can be used for the second threshold value based on the length of reads at issued in the implementation. Moreover, there is no requirement that the first threshold and the second threshold use the same threshold value.

### “YES” Branch of Stage 314

If it is determined by the compression engine 120 that the number of undisclosed bases “N” in the imperfectly mapped read exceeds the second threshold, then the compression engine 120 can determine that the imperfectly mapped read is to be encoded using the second encoding module 310—e.g., included in the compression engine 120—to encode the imperfectly mapped read using the second encoding process. The second encoding process includes initially encoding each nucleotide or base individually regardless of whether said nucleotide or base is aligned or not. In some implementations, because the compression engine 120 determined that the number of undetermined bases “N” exceeded the second threshold at stage 314, the compression engine 120 can use the second encoding module to encode the read into 4 bits 310a using the second encoding process. Once a read is encoded using the second encoding process 310 using 4-bit encoding 310a, the compression engine 120 can store the encoded read in a memory or other storage device at stage 322. The compression engine 120 can determine, at stage 324, whether there is another, sequentially ordered aligned read that is to be compressed. And, if there is another, sequentially ordered aligned read that is to be compressed, the compression engine 120 can execute the operations of stage 302 in order to obtain the next sequentially ordered aligned read record and execute the process 300 again. The compression engine 120 can continue to iteratively execute process 300 until no more sequentially ordered aligned read records are identified at stage 324. Upon such a determination, the process 300 can terminate at stage 326.

### “NO” Branch of Stage 314

If the compression engine 120 determines, during stage 314, that the number of undisclosed bases “N” in the imperfectly mapped read does not exceed the second threshold, then the compression engine 120 can determine that the imperfectly mapped read is to be encoded using the second encoding module 310—e.g., included in the compression engine 120—to encode the imperfectly mapped read using the second encoding process. The second encoding process includes individually encoding each nucleotide or base regardless of whether said nucleotide or base is aligned or not. In some implementations, because the compression engine 120 determined that the number of undetermined bases “N” did not exceed the second threshold at stage 314, the compression engine 120 can use the second encoding module to encode the read into 2 bits 310b using the second encoding process. Once a read is encoded using the second encoding process 310 using 2-bit encoding 310b, the compression engine 120 can store the encoded read in a memory or other storage device at stage 322. The compression engine 120 can determine, at stage 324, whether there is another, sequentially ordered aligned read that is to be compressed. And, if there is another, sequentially ordered aligned read that is to be compressed, the compression engine 120 can execute the operations of stage 302 in order to obtain the next sequentially ordered aligned read record and execute the process 300 again. The compression engine 120 then can continue to iteratively execute process 300 until no more sequentially ordered aligned read records are identified at stage 324. Upon such a determination, the process 300 can terminate at stage 326.

### “NO” Branch of Stage 306

If the compression engine 120 determines at stage 306 that the number of differences between the imperfectly mapped read and the reference sequence does not exceed a first threshold, then the compression engine 120 can continue execution of the process 300 at stage 308. At stage 308, the compression engine 120 can determine whether the imperfectly mapped read includes more than a second threshold number of undetermined bases “N.”

### “YES” Branch of Stage 308

If the compression engine 120 determines, at stage 308, that the number of undisclosed bases “N” in the imperfectly mapped read exceeds the second threshold, then the compression engine 120 can determine at stage 308 that the imperfectly mapped read is to be encoded using the second encoding module 310 to encode the imperfectly mapped read using the second encoding process. In some implementations, because the compression engine 120 determined that the number of undetermined bases “N” exceeded the second threshold at stage 308, the compression engine 120 can use the second encoding module to encode the read into 4 bits 310a using the second encoding process. Once read is encoded using the second encoding process 310 using 4-bit encoding 310a, the compression engine 120 can store the encoded read in a memory or other storage device at stage 322. The compression engine 120 can determine, at stage 324, whether there is another, sequentially ordered aligned read that is to be compressed. And, if there is another, sequentially ordered aligned read that is to be compressed, the compression engine 120 can execute the operations of stage 302 in order to obtain the next sequentially ordered aligned read record and execute the process 300 again. The compression engine 120 can continue to iteratively execute process 300 until no more sequentially ordered aligned read records are identified at stage 324. Upon such a determination, the process 300 can terminate at stage 326.

### “NO” Branch of Stage 308

If the compression engine 120 determines, at stage 308, that the imperfectly mapped read includes a number of undetermined bases “N” that does not satisfy the second threshold, then compression engine 120 can use a third encoding module 312 to encode the imperfectly mapped read using a third encoding process described in this document.

Once the read is encoded using the third encoding process of the third encoding module 312, the compression engine 120 can store the encoded read in a memory or other storage device at stage 322. The compression engine 120 can determine, at stage 324, whether there is another, sequentially ordered aligned read that is to be compressed. And, if there is another, sequentially ordered aligned read that is to be compressed, the compression engine 120 can execute the operations of stage 302 in order to obtain the next sequentially ordered aligned read record and execute the process 300 again. The compression engine 120 can continue to iteratively execute process 300 until no more sequentially ordered aligned read records are identified at stage 324. Upon such a determination, the process 300 can terminate at stage 326.

### “Read Perfectly Mapped” Branch of Stage 304

Alternatively, if it is determined at stage 304 that the read corresponding to the obtained read record is a perfectly mapped read, then the compression engine 120 can determine, at stage 316, whether the perfectly mapped read includes a number of undetermined bases “N” that exceeds a second threshold value. In some implementations, the second threshold value can also be equal to 31. However, like the first thresholds, the present disclosure is not limited to a second threshold value of 31. Instead, any number value, including higher values than 31, can be used for the second threshold value based on the length of reads at issue in the implementation. Moreover, there is no requirement that the first threshold and the second threshold use the same threshold value.

### “NO” Branch of Stage 316

If the compression engine 120 determines at stage 316 that the perfectly mapped read does not include more than a second threshold number of undetermined bases “N,” then the compression engine 120 can determine to encode the read using the first encoding module 322 using a first encoding process described in this document. If the perfectly mapped read does not include any undetermined bases “N,” then the first encoding module 322 executes a first encoding process that is the same as the first encoding process described in this document. Alternatively, if the perfectly mapped read includes one or more “N,” then the first encoding module 322 encodes the perfectly mapped read using the first encoding process. In addition, in the particular implementation where the perfectly mapped read includes one or more N (but less than a second threshold number of N), the first encoding module 318 can also store a list of positions on the read for the undetermined bases N.

Different encoding processes can use distinct sets of descriptors. For example, a first and third encoding processes can use distinct sets of descriptors. Each set of descriptors univocally represents the reads associated to the corresponding encoding process, each of the first and third encoding processes being a reduced information entropy encoding process. More precisely, the third encoding process can include a first encoding process and a second encoding process. The imperfectly mapped reads that are determined to be globally mapped during the process 300 are encoded according to the first encoding process. The imperfectly mapped reads that are determined to be locally mapped are encoded according to the second encoding process. The first and second encoding processes comprise distinct sets of descriptors, each set of descriptors univocally representing the reads associated to the corresponding encoding process as described in this document.

The alignment information encoded for each read, and which enables the reconstruction of the whole read sequence during the decompression of the data, can depend on the corresponding encoding process or process used for said read.

For example, in some implementations, a first set of descriptors used for the first encoding process can include: (i) the absolute starting position of the perfectly mapped read with respect to the reference sequence (encoded on 16 or 32 bits), and (ii) the length of the read (encoded with differential coding relative to the length of the previous read, with variable length code ranging from 2 bits to 34 bits).

By way of another example, in some implementations, a second set of descriptors used for the second encoding process can include: (i) the absolute starting position of the imperfectly mapped read with respect to the reference sequence (encoded on 16 or 32 bits), (ii) the length of the read (encoded with differential coding relative to the length of the previous read, with variable length code ranging from 2 bits to 34 bits), and (iii) a list of the mismatches of the read.

By way of another example, in some implementations, a third set of descriptors used for the third encoding process can include: (i) the absolute starting position of the imperfectly mapped portion of the read with respect to the reference sequence—also called local alignment starting position (encoded on 16 or 32 bits), (ii) the length of the read (encoded with differential coding relative to the length of the previous read, with variable length code ranging from 2 bits to 34 bits), (iii) a list of the mismatches of the read, and (iv) the length of the clipped portions of the read that are not part of the alignment (encoded on 8 bits for each clipped portion).

Preferably, the list of mismatches which is encoded in the first and second processes can include a header. For example, in some implementations, the header can be encoded using a bit flag and be encoded on one byte. In such implementations, the five first bits of the one-byte header can be used to encode the number of mismatches contained in the read. In implementations where the threshold value is equal to 31, the number of mismatches can range between 0 and 31. One bit of the one-byte header can be used to encode whether the imperfectly mapped read is globally or locally mapped. Another bit of the one-byte header can be used to encode whether or not the 2-bit mode is activated for the second encoding process. The last bit of the one-byte header can be used to encode whether or not the 4-bit mode is activated for the second encoding process. In some implementations, for each read encoded according to the second encoding process during the encoding stage 12, the clipped portions of said read (e.g., those portions that are not part of the local alignment) are concatenated, and each nucleotide or base of said clipped portions is individually encoded. In some implementations, each nucleotide or base of such clipped portions of the read is individually encoded on 2 bits.

In some implementations, each mismatch encoded in the list of mismatches of an imperfectly mapped read (e.g., encoded according to the first or second encoding process) can be encoded on 1 byte. More precisely, each mismatch of an imperfectly mapped read that is to be encoded according to the first or second encoding process may be encoded as follows: (i) the two first bits of the byte are used to encode the alternate nucleotide or base present in the read instead of the corresponding reference nucleotide or base in the reference sequence, (ii) the six last bits are used to encode the position of the mismatch in the reference sequence, said position being computed as an offset from the previous mismatch of the read. This computed position can be a relative position of the mismatch, except for the first mismatch of the read for which the absolute position is encoded. The range of this offset, which is encoded on 6 bits, can therefore be [0-63].

The encoded, or compressed, record that result from the completion of the processes can be stored in a memory or other storage device of the control unit 104. In some implementations, this encoded, or compressed, record can be stored in the memory or other storage device of the control unit 104 in a manner that maintains the sequence ordering of the read records. This helps to ensure that compression of the aligned read records is lossless since even the initial sequence ordering of the aligned read records is preserved.

Once a read—e.g., a read of the sample 118—is encoded using the first encoding module 318, the compression engine 120 can store the encoded read in a memory or other storage device at stage 322. The compression engine 120 can determine, at stage 324, whether there is another, sequentially ordered aligned read that is to be compressed. And, if there is another, sequentially ordered aligned read that is to be compressed, the compression engine 120 can execute the operations of stage 302 in order to obtain the next sequentially ordered aligned read record and execute the process 300 again. The compression engine 120 can continue to iteratively execute process 300 until no more sequentially ordered aligned read records are identified at stage 324. Upon such a determination, the process 300 can terminate at stage 326.

### “YES” Branch of Stage 316

However, if the compression engine 120 determines at stage 316 that the read does include more than a second threshold number of undetermined bases “N,” then the compression engine 120 can use the second encoding module 310 to encode the read into 4 bits 310a using the second encoding process described in this document. Once a read is encoded using the second encoding process 310 using 4-bit encoding 310a, the compression engine 120 can store the encoded read in a memory or other storage device at stage 322. The compression engine 120 can determine, at stage 324, whether there is another, sequentially ordered aligned read that is to be compressed. And, if there is another, sequentially ordered aligned read that is to be compressed, the compression engine 120 can execute the operations of stage 302 in order to obtain the next sequentially ordered aligned read record and execute the process 300 again. The compression engine 120 can continue to iteratively execute process 300 until no more sequentially ordered aligned read records are identified at stage 324. Upon such a determination, the process 300 can terminate at stage 326.

### “Unmapped Read” Branch of Stage 304

Alternatively, if it is determined at stage 304 that the read corresponding to the obtained read record is an unmapped read, then the compression engine 120 can determine, at stage 320, whether the unmapped read includes a number of undetermined bases “N” that exceeds a second threshold value. In some implementations, the second threshold value can also be equal to 31. However, like the first thresholds, the present disclosure is not limited to a second threshold value of 31. Instead, any number value, including higher values than 31, can be used for the second threshold value based on the length of reads at issued in the implementation. Moreover, there is no requirement that the first threshold and the second threshold use the same threshold value.

### “NO” Branch of Stage 320

If the compression engine 120 determines at stage 320 that the unmapped read does not include more than a second threshold number of undetermined bases “N,” then the compression engine 120 can determine to encode the read using the second encoding module 310 using a second encoding process as described in this document. In some implementations, because the compression engine 120 determined that the number of undetermined bases “N” did not exceed the second threshold at stage 320, the compression engine 120 can use the second encoding module to encode the read into 2 bits 310b using the second encoding process. Once a read is encoded using the second encoding process 310 using 2-bit encoding 310b, the compression engine 120 can store the encoded read in a memory or other storage device at stage 322. The compression engine 120 can determine, at stage 324, whether there is another, sequentially ordered aligned read that is to be compressed. And, if there is another, sequentially ordered aligned read that is to be compressed, the compression engine 120 can execute the operations of stage 302 in order to obtain the next sequentially ordered aligned read record and execute the process 300 again. The compression engine 120 can continue to iteratively execute process 300 until no more sequentially ordered aligned read records are identified at stage 324. Upon such a determination, the process 300 can terminate at stage 326.

### “YES” Branch of Stage 320

However, if the compression engine 120 determines at stage 320 that the unmapped read does include more than a second threshold number of undetermined bases “N,” then the compression engine 120 can use the second encoding module 310 to encode the read into 4 bits 310a using the second encoding process as described in this document. Once the read is encoded using the second encoding process 310 using 4-bit encoding 310a, the compression engine 120 can store the encoded read in a memory or other storage device at stage 322. The compression engine 120 can determine, at stage 324, whether there is another, sequentially ordered aligned read that is to be compressed. And, if there is another, sequentially ordered aligned read that is to be compressed, the compression engine 120 can execute the operations of stage 302 in order to obtain the next sequentially ordered aligned read record and execute the process 300 again. The compression engine 120 can continue to iteratively execute process 300 until no more sequentially ordered aligned read records are identified at stage 324. Upon such a determination, the process 300 can terminate at stage 326.

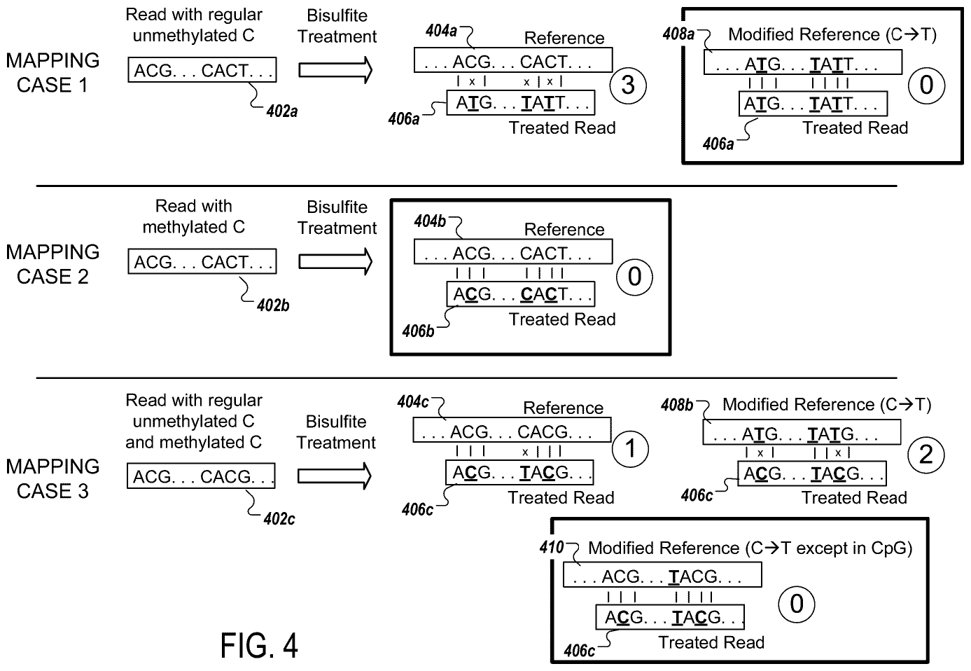

FIG. 4 is a diagram showing an example of comparing genomic data reads to references. Three cases are shown: a first case where a read includes unmethylated C, a second case where a read includes methylated C, and a third case where a read includes unmethylated and methylated C. The example mappings are meant to be illustrative and do not show all elements of all types of comparison—e.g., hash table generation and look up as described in this document.

In each case, the read is treated using a bisulfite treatment. The bisulfite treatment is chemical transformation that transforms C to T as mentioned in this document. FIG. 4 shows how different modified references can generate fewer mismatches than an unmodified reference. For example, in mapping case 1, the read 402a is treated to generate treated read 406a—where the unmethylated Cs are transformed in Ts. The treated read 406a can be compared to reference 404a which is unmodified from a typical genome reference (e.g., no C to T transformations, among others). Comparison can be performed by, e.g., the comparison engine 114 of FIG. 1. The treated read 406a compared to the reference 404a results in 3 mismatches. A comparison with a modified reference 408a—where Cs are transformed to Ts—results in 0 mismatches. In the context of FIG. 1, the control unit 104 could select modified reference 408a for compressing a mapping of the treated read 406a because it has fewer mismatches than the other reference used.

In mapping case 2, the read 402b is treated to generate treated read 406b—where the methylated Cs are not transformed to Ts. The treated read 406b can be compared to reference 404b which is unmodified from a typical genome reference (e.g., no C to T transformations, among others). Comparison can be performed by, e.g., the comparison engine 114 of FIG. 1. The treated read 406b compared to the reference 404b results in 0 mismatches. In the context of FIG. 1, the control unit 104 could select reference 404b for compressing a mapping of the treated read 406b because it has fewer mismatches than the other reference used (other references used not shown). In some implementations, only a single reference is used.

In mapping case 3, the read 402c is treated to generate treated read 406c—where some instances of C in the read are transformed to T and others are not corresponding to whether or not the given C is methylated or unmethylated. The treated read 406c can be compared to reference 404c which is unmodified from a typical genome reference similar to reference 404a and 404b described. Comparison can be performed by, e.g., the comparison engine 114 of FIG. 1. The treated read 406c compared to the reference 404c results in 1 mismatch. The treated read 406c can also be compared to other references—e.g., modified reference 408b where Cs are transformed to T and modified reference 410 where Cs are transformed to T except in CpG regions. The former comparison results in 2 mismatches while the latter results in 0. In the context of FIG. 1, the control unit 104 could select modified reference 410 for compressing a mapping of the treated read 406c because it has fewer mismatches than the other reference used.

FIG. 5 is a diagram showing an example variant call format (VCF) code snippet. The diagram shows “REF” and “ALT” columns which exist in prior versions of VCF and are not suitable for reporting methylation—e.g., because such existing columns require specific data formats that are incompatible with methylation reporting, such as requirements to only include “A”, “C”, “G”, “T”, “N”, or “.” characters. The diagram includes “INFO”, “FORMAT”, and genotype (e.g., “NA00001”, “NA00002”, and “NA00003”) columns which can be used to report methylation information with new tags. The tag in the “INFO” column can be used to mark nucleotides on REF and ALT alleles, for which methylation level is reported. The tag in the “FORMAT” column with respective content in the genotype (“NA00001”, “NA00002”, and “NA00003”) fields can be introduced to report various methylation information, such as methylation level (μ), total number of reads covering a cytosine (n), difference of methylation levels on opposite strands for a CpG dinucleotide (δ), probability that this difference is zero (p), hemi-methylation level (h) with respective probability (pₕ) that hemi-methylation level is zero.

By combining methylation information with variant identification in a single data structure, the enhanced VCF effectively compresses the amount of data required to store, display, or process variant information with an indication of methylation type. The enhanced VCF improves variant methylation compression by (i) enabling storage of methylation per variant—e.g., 5-methylcytosine, 5-hydroxymethylcytosine, N6-methyladenine, N4-methylcytosine, or a combination of these among others can be stored together with other variant information to reduce duplication of associated genetic mapping information, (ii) enabling storage of multiple methylation states—e.g., for one or more variants, (iii) enabling storage of cytosine methylation information that overlaps with variants—e.g., in a CpG region, (iv) enabling simultaneous reporting of variant and methylation to reduce data transferring, storage, and related processing, and (v) enabling storage of allele-specific methylation data.

A number of implementations have been described. Nevertheless, it will be understood that various modifications may be made without departing from the spirit and scope of the disclosure. For example, various forms of the flows shown above may be used, with operations re-ordered, added, or removed.

Implementations of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Implementations of the subject matter described in this specification can be implemented as one or more computer programs, i.e., one or more modules of computer program instructions encoded on a tangible non-transitory program carrier for execution by, or to control the operation of, data processing apparatus. Alternatively, or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus. The computer storage medium can be a machine-readable storage device, a machine-readable storage substrate, a random or serial access memory device, or a combination of one or more of them.

The term “data processing apparatus” refers to data processing hardware and encompasses all kinds of apparatus, devices, and machines for processing data, including by way of example a programmable processor, a computer, or multiple processors or computers. The apparatus can also be or further include special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, a module, a software module, a script, or code, can be written in any form of programming language, including compiled or interpreted languages, or declarative or procedural languages, and it can be deployed in any form, including as a stand-alone program or as a module, component, subroutine, or other unit suitable for use in a computing environment. A computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application-specific integrated circuit).

Computers suitable for the execution of a computer program include, by way of example, general or special purpose microprocessors or both, or any other kind of central processing unit. Generally, a central processing unit will receive instructions and data from a read-only memory or a random-access memory or both. The essential elements of a computer are a central processing unit for performing or executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a smart phone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, implementations of the subject matter described in this specification can be implemented on a computer having a display device, e.g., LCD (liquid crystal display), OLED (organic light emitting diode) or other monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some implementations, a server transmits data, e.g., a Hypertext Markup Language (HTML) page, to a user device, e.g., for purposes of displaying data to and receiving user input from a user interacting with the user device, which acts as a client. Data generated at the user device, e.g., a result of the user interaction, can be received from the user device at the server.

An example of one such type of computer is shown in FIG. 1, which shows a schematic diagram of a system 100. The system 100 can be used for the operations described in association with any of the computer-implemented methods described previously, according to one implementation. The system 100 can include a processor, a memory, a storage device, and an input/output device (e.g., as part of the control unit 104 or elements for computing or storage communicably connected to the control unit 104). Each of the components can be interconnected using a system bus. The processor is capable of processing instructions for execution within the system 100. In one implementation, the processor is a single-threaded processor. In another implementation, the processor is a multi-threaded processor. The processor is capable of processing instructions stored in memory or obtained by the control unit 104 to display graphical information for a user interface on an input/output device.

While this specification contains many specific implementation details, these should not be construed as limitations on the scope of what may be claimed, but rather as descriptions of features that may be specific to particular implementations. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the implementations described above should not be understood as requiring such separation in all implementations, and it should be understood that the described program components and systems can generally be integrated together in a single software product or packaged into multiple software products.

In each instance where an HTML file is mentioned, other file types or formats may be substituted. For instance, an HTML file may be replaced by an XML, JSON, plain text, or other types of files. Moreover, where a table or hash table is mentioned, other data structures (such as spreadsheets, relational databases, or structured files) may be used.

Particular implementations of the invention have been described. Other implementations are within the scope of the following claims. For example, the operations recited in the claims, described in the specification, or depicted in the figures can be performed in a different order and still achieve desirable results. In some cases, multitasking and parallel processing may be advantageous.

1. A method for compressing genomic reads, the method comprising:
obtaining a genomic data read;
mapping the read to a plurality of different candidate reference genomes;
selecting one of the candidate reference genomes based on the mapping;
performing reference-based compression of the read using the selected reference genome; and
storing the compressed genomic data read.

2. The method of claim 1, wherein the plurality of different candidate reference genomes includes two or more candidate reference genomes that are modifications of a single genomic reference.

3. The method of claim 1, wherein the plurality of different candidate reference genomes comprises a candidate reference genome that includes nucleotide sequences of a methylome.

4. The method of claim 3, wherein the candidate reference genome that includes nucleotide sequences of the methylome includes portions of the methylome where cytosine is converted to thymine.

5. The method of claim 4, wherein the portions of the methylome where cytosine is converted to thymine are not CpG regions.

6. The method of claim 3, wherein the candidate reference genome that includes nucleotide sequences of the methylome includes portions of the methylome where guanine is converted to adenine.

7. The method of claim 6, wherein the portions of the methylome where guanine is converted to adenine are not CpG regions.

8. The method of claim 1, wherein storing the compressed genomic data read comprises:
storing an indication of a number of mismatches between nucleotides of the read and the selected reference genome.

9. The method of claim 1, wherein storing the compressed genomic data read comprises:
storing an indication of an offset that indicates a position of the genomic data read relative to the selected reference genome.

10. The method of claim 9, wherein the offset indicates a number of nucleotides from a beginning of the selected reference genome to a start of the genomic data read.

11. The method of claim 9, comprising:
generating a combined reference using the plurality of different candidate reference genomes, wherein the offset is equal to an offset between the genomic data read and the selected reference genome plus a length of one or more of the plurality of different candidate reference genomes.

12. The method of claim 1, wherein selecting the one of the candidate reference genomes based on the mapping comprises:
determining a number of mismatches between the genomic data read and the plurality of different candidate reference genomes.

13. The method of claim 12, comprising:
selecting the selected reference genome based on a minimum mismatch value from the number of mismatches between the genomic data read and the plurality of different candidate reference genomes.

14. The method of claim 1, wherein storing the compressed genomic data read comprises:
generating encoded data representing the genomic data read; and
storing the encoded data representing the genomic data read in a hash table.

15. One or more non-transitory computer storage media encoded with instructions that, when executed by one or more computers, cause the one or more computers to perform operations comprising:
obtaining a genomic data read;
mapping the read to a plurality of different candidate reference genomes;
selecting one of the candidate reference genomes based on the mapping;
performing reference-based compression of the read using the selected reference genome; and
storing the compressed genomic data read.

16. The media of claim 15, wherein the plurality of different candidate reference genomes includes two or more candidate reference genomes that are modifications of a single genomic reference.

17. The media of claim 15, wherein the plurality of different candidate reference genomes comprises a candidate reference genome that includes nucleotide sequences of a methylome.

18. A system comprising one or more computers and one or more storage devices on which are stored instructions that are operable, when executed by the one or more computers, to cause the one or more computers to perform operations comprising:
obtaining a genomic data read;
mapping the read to a plurality of different candidate reference genomes;
selecting one of the candidate reference genomes based on the mapping;
performing reference-based compression of the read using the selected reference genome; and
storing the compressed genomic data read.

19. The method of claim 1, comprising:
storing data indicating an associated methylation state for one or more variants, the one or more variants determined based on mapping the read to at least one additional reference genome.

20. The method of claim 19,
wherein storing the data indicating the associated methylation state for the one or more variants comprises storing the data indicating the associated methylation state with data indicating the one or more variants in a single data structure.
